(19) United States
(12) Patent Application Publication
(10) Pub. No.: US 2011/0060409 A1
(43) Pub. Date: Mar. 10, 2011

(54) Optics and IOLs for Inhibiting cell migration and reduce optic edge dysphotopsia

(76) Inventor: VALDEMAR PORTNEY, Newport Coast, CA (US)

(21) Appl. No.: 12/857,445

(22) Filed: Aug. 16, 2010

Related U.S. Application Data
(60) Provisional application No. 61/239,928, filed on Sep. 4, 2009.

(51) Int. Cl. A61F 2/16

(52) U.S. Cl. 623/6.39

(57) ABSTRACT

An intraocular lens implantable in an eye includes an optic for placement in the capsular bag of the eye and for directing light toward the eye's retina. The optic has a central optical axis, an anterior surface, an opposing posterior surface and optic peripheral edge surface between the surfaces. The peripheral edge includes regions of a substantially continuous configuration with radii are such that their optical centers either within the optic or outside the optic and also being substantially smaller the radius of the optic of equivalent dimension but circular shape of substantially constant radius. The peripheral edge surface has a substantially flat configuration in the direction between the surfaces. The intersection of the peripheral edge surface and at least one of the anterior surface and the posterior surface, forms a discontinuous sharp corner edge.

The present application claimed priority from U.S. Provisional Patent Application Ser. No. 61/239,928. This application is incorporated herein in its entirety by this specific reference thereto.

This invention relates to intraocular lenses (IOLs) and, more particularly, to IOL which inhibits cell migration from the eye onto an optical zone of the IOL and reduce optic edge dysphotopsia in the eye.

An intraocular lens is commonly used to replace the natural lens of a human eye for aphakia treatment. It is common practice to implant an IOL in a region of the eye known as the capsular bag. There are two problems with many IOLs following implantation in the capsular bag:
(1) Reduction of image contrast caused by the cells migration to the optical zone of the IOL
(2) dysphotopsia caused by light reflecting off the peripheral edge of the IOL optic,

A common treatment for this condition is to use a laser to destroy the cells and a central region of the posterior capsular bag. Although this treatment is effective and is usually done when the vision diminishes to unacceptable level. There is also cost associated with the laser treatment. In addition, it also may result in the IOL positional shift in the capsular bag thus affecting IOL optical performance.

So-called “square-edged IOL” design with sharp transitions between the IOL edge and the surfaces seems to help in delaying the cell migration. Another benefit observed was a tight capsule shrink-wrap effect with the square-edged IOLs with the fibrotic ring, allowing minimal IOL shift. The round-edged IOLs on the other hand, tends to decenter and rotate more. This consideration is particularly important for tonic IOLs where the lens meridional orientation inside the eye is the critical factor to providing acceptable performance for cylinder correction. It is also important consideration for Accommodating IOLs which rely on the lens positional stability.

Thus, while square-edged IOLs are more helpful in preventing the cell migration they manifest the issue of light reflection off the optic edge surface resulting in the reports of dysphotopsia by some patients. The dysphotopsia can be an annoyance up to the point of requesting the IOL removal and replacement for other type of IOL. The round-edged IOLs on the other hand, are known to minimize edge dysphotopsia as compared with square-edged IOLs. This is due diversion of the light reflected from the rounded optic edge over the wider area of the retina thus reducing the light intensity.

U.S. Pat. No. 6,162,249 describes the IOLs with optic peripheral edge having a substantially continuous curved configuration relative to the central optical axis in order to maintain square-edge IOL feature to have a sharp edge at the junction between peripheral edge surface and optical surface of the IOL and, as a result, to inhibit cell migration together with the reduction of edge glare or dysphotopsia.

The issue with the proposed IOLs in the U.S. Pat. No. 6,162,249 is difficulty of manufacturing such an peripheral edge shape thus limiting the edge shape to certain configurations which might not be optimal for reduction in edge dysphotopsia. The common process to produce optic peripheral edge of the optic is to use milling where the endmill cuts out the lens shape from the button or cut the corresponding shape in the mold for the optic molding. To produce the optic peripheral edge shape that is not parallel to the optical axis would require a specially shaped endmill and its precise location along the vertical axis parallel to the optical axis or additional fabrication process to polish out the optic periphery edge to a desirable configuration.

Thus, it would be advantageous to introduce IOLs which inhibit growth of cells at the IOL placed in the capsular bag and further optimize the optic peripheral edge shape for dysphotopsia reduction and also to allow using conventional manufacturing processes.

### SUMMARY OF THE INVENTION

New IOLs have been discovered that combine ease of manufacturing with an unlimited configurations of the optic peripheral edge to diverge the reflected light over the wide area of the retina. Such IOLs are effective to inhibit cell migration due to the presence of sharp discontinuity between the optical peripheral edge surface and lens anterior or posterior surface. The optic peripheral edge surface can maintain flat shape parallel to the optical axis of the optic and as such, easy to manufacture with a commonly available endmill and utilizing conventional manufacturing processes.

The IOLs in accordance with the present invention includes an optic having a central optical axis, an optic anterior surface, an opposing optic posterior surface and an optic peripheral edge between the optic surfaces. The optic of the IOL is adapted for placement in the capsular bag of the eye and for focusing light toward the eye's retina. The IOLs in accordance with the present invention further include at least one fixation member commonly called a haptic, and preferably two fixation members, connected to the optic for fixation the IOL in the eye. In general, the IOL may include a plate shape haptc or include the optic consisting of several lenses.

The optic peripheral edge of the present IOLs include an undulated or periodic segment of a substantially continuous configuration in the direction around the central optical axis of the optic, meaning in the plane perpendicular to the optical axis of the optic. A preferable embodiment includes the entire optic peripheral edge having a substantially continuous curved configuration of variable curvature i.e., undulated or periodic in the plane perpendicular to the optical axis thus maintaining the cylinder shape of the optic peripheral edge substantially around the optic.

The variable curvature of substantially continuous curved configuration includes radii with their optical centers either within the optic and outside the optic and also being substantially smaller the radius of the optic of equivalent dimension but circular shape of substantially constant radius. The presence of the regions of smaller radii results in broad divergence of the reflected from the optic peripheral edge light over much larger retinal area than in the circular shape optic with substantially constant radius of the flat peripheral edge. Therefore, the present IOLs lead to reduced edge dysphotopsia in the eye relative to the dysphotopsia gained with a substantially identical IOL with optic shape of substantially constant radius and peripheral edge surface of flat peripheral edge.

Thus, optic peripheral edge of the disclosed IOL maintains discontinues sharp corner (corner edge) between the optic peripheral edge surface and optical surface, so called square-edged shape as the prior art square-edged IOL with the optic of a substantially constant radius. In general, the flat peripheral edge between anterior and posterior surfaces of the invented IOL may be tilted from preferable parallel to central optical axis configuration to up about 45 degrees to the central optical axis. The preferable embodiment maintains discontinuous sharp corners between the optical peripheral edge and both anterior and posterior optical surfaces.

### BRIEF DESCRIPTION OF THE FIGURES

FIG. 1 is a partial cross-sectional view of an optic of a prior art IOL.

FIG. 2 is a partial cross-sectional view of another optic of a prior art IOL.

FIG. 3 is a plane view of one embodiment of IOL in accordance with the present invention superimposed over the equivalent IOL shape but with circular optic of substantially constant radius.

FIG. 4 is a cross-sectional view taken generally along line 4-4 of FIG. 3.

FIG. 5 is a plane of a segment taken from the IOL of FIG. 3.

### DETAILED DESCRIPTION

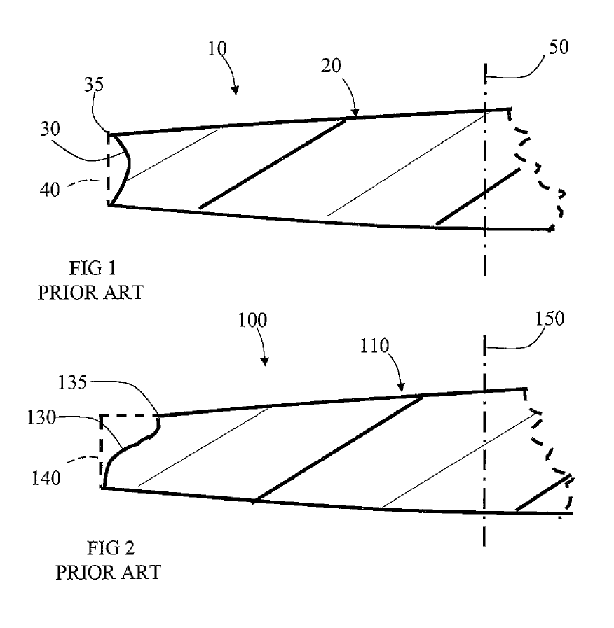

FIG. 1 illustrates a partial cross-sectional view of an optic 10 of a prior art IOL which has curved optic peripheral edge 30 along the central optical axis 50 as compared with a flat optic peripheral edge 40 (dashed line) that is parallel to the optical axis 50. The optic peripheral edge maintains sharp corner edge 35 with anterior or posterior surface 20 similar to the flat optic peripheral edge 40 to provide inhibition of cell growth. Curved optic peripheral edge 30 provides a reduction in dysphotopsia as compared with flat circular peripheral edge 40 of the optic with substantially constant radius.

FIG. 2 illustrates a partial cross-sectional view of an optic 100 of another prior art IOL which has more complex curved optic peripheral edge 130 along the central optical axis 150 as compared with the flat optic peripheral edge 140 that is parallel to the optical axis 150. The optic peripheral edge also maintains sharp corner edge 135 with anterior or posterior surface 110 similar to the flat optic peripheral edge 140 to provide inhibition of cell growth. Curved optic peripheral edge 130 also provides a reduction in dysphotopsia as compared with flat circular peripheral edge 140 of the optic with substantially constant radius.

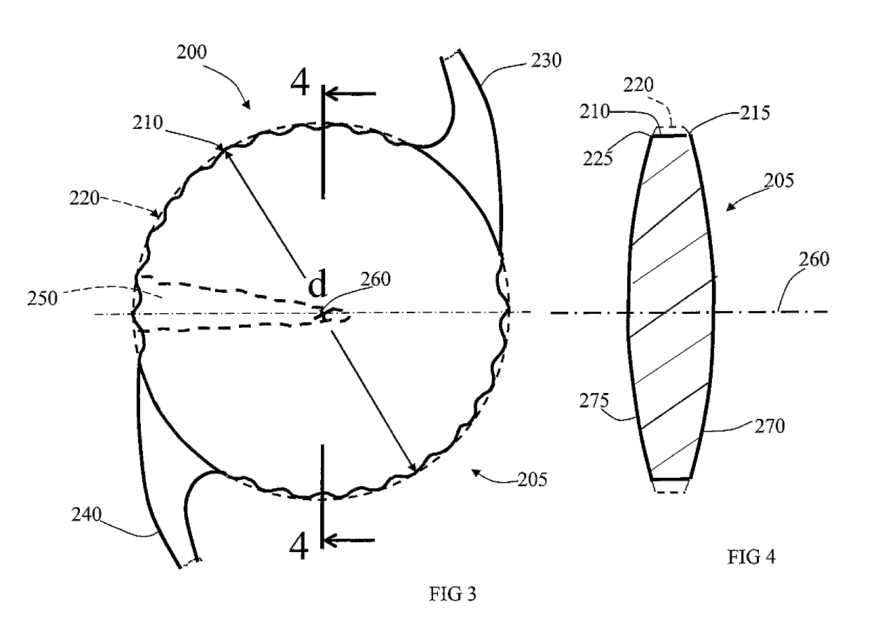

FIG. 3 illustrates a plane view of an embodiment of IOL 200 in accordance with the present invention. In this embodiment, the optic 205 incorporates an undulated or periodic peripheral edge 210 around the optic except at the location of the fixation members 230 and 240. The optic 205 of optic diameter “d” is superimposed over the optic of constant radius that is half of “d” and with circular peripheral edge 220. The optic 205 has central optical axis 260 which is also the optical axis of the optic with circular peripheral edge 220. The optic may have an oval shape and the corresponding diameter is the average diameter of smallest and largest optic diameters.

In this embodiment, the optic 205 is circular shape in plan, with an undulated or periodic peripheral edge 210 and bi-convex shape with the optical axis 260. However, this configuration is clearly illustrative as other configurations and shapes may be employed.

The optic 205 may be constructed of any of the commonly utilized IOL materials used for rigid optics, such as polymethylmethacrylate (PMMA), or commonly employed materials used for deformable optics, such as silicone polymeric materials, acrylic polymeric materials, hydrogel-forming polymeric materials and the like.

Two fixation members 230 and 240 in this embodiment are generally C or J-shaped and are connected to the optic 205. However, this is purely illustrative of the fixation members 230 and 240 as the fixation members may be of other configurations and numbers.

The segment 250 of the optic 205 that includes variable radii of the peripheral edge 210 and the central optical axis 260 is referenced to in order to explain the invented IOL in more details in the following figure.

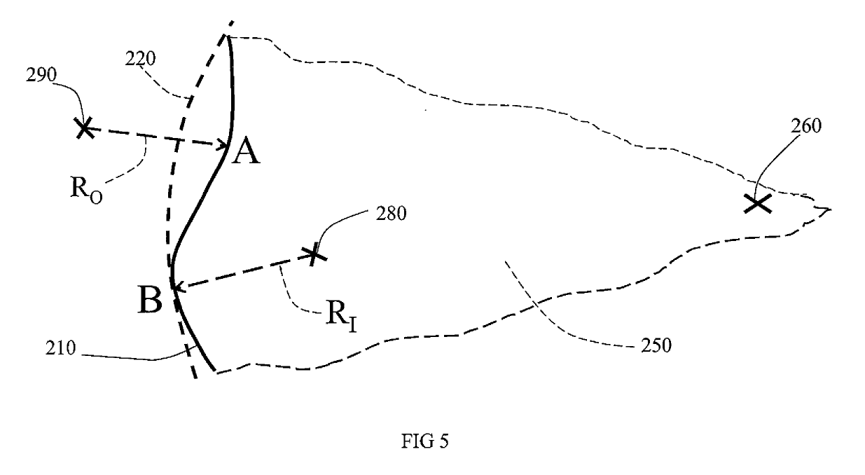

FIG. 4 illustrates a cross-sectional view taken generally along line 4-4 of FIG. 3. The preferred embodiment of peripheral edge 210 of the optic 205 is shown as flat and parallel to the optical axis 260 and include discontinuous sharp edges 215 and 225 between the peripheral edge surface and anterior and posterior surfaces 270 and 275. The peripheral edge 220 of the optic of substantially constant radius of half diameter “d” shown on FIG. 3 is also included for the reference.

In general, the inhibition of the cell and reduction of the edge glare per the invented IOL can be achieved with the peripheral edge being substantially flat with discontinuous sharp corner edge forming between the peripheral edge surface and only one of the anterior and posterior surfaces. The substantially flat peripheral edge surface can be tilted to the optical axis by up to about 45 degrees.

FIG. 5 explains the invented IOL in more details on the example of the segment 250 of FIG. 3. The peripheral edge 210 within the segment 250 of the optic 205 includes the regions “A” and “B” of substantially continuous curvatures which include radii “R_{I}” and “R_{O}” somewhere within the corresponding regions “A” and “B” correspondently. The central optical axis 260 and the peripheral edge 220 of substantially constant radius with the same optical axis 260 are shown on the figure. The radius “R_{I}” has the center of radius 280 within the optic 205 and is substantially smaller the distance between peripheral edge 220 and optical axis 260. The radius “R_{O}” has the center of radius 290 outside the optic 205 and is also substantially smaller the distance between peripheral edge 220 and optical axis 260.

The segment 250 of the preferred embodiment includes the regions “A” and “B” that are connected and repeated substantially over the whole peripheral edge 210 of the optic 205 in FIG. 3. In general, the regions can be located at substantially different parts of the optic peripheral edge and without a repetition.

What is claimed is:

1. An optic suitable for use in an intraocular lens, the optic comprising:
an anterior surface;
a posterior surface;
a central optical axis; and
a peripheral edge surface having undulated segment, the edge surface intersecting at least one of the anterior and posterior surfaces to form a discontinuous sharp corner edge between the undulated peripheral surface and at least one of the anterior and posterior surfaces.

2. The optic according to claim 1 wherein the undulated segment of the peripheral edge surface is flat.

3. The optic according to claim 2 wherein the flat undulated segment of the peripheral edge surface is parallel to a central optic axis of the optic.

4. The optic according to claim 1 wherein the flat undulated peripheral edge surface includes at least one trough and at least one crest defined by radii substantially smaller than an optic radius.

5. The optic according to claim 1 wherein the undulated segment includes an entire peripheral edge surface.

6. An optic suitable for use in an intraocular lens, the optic comprising:
an anterior surface;
a posterior surface;
a central optical axis; and
a periodical peripheral edge surface intersecting at least one of the anterior and posterior surfaces to form a discontinuous sharp corner edge between the undulated peripheral edge surface and at least one of the anterior and posterior surfaces.

7. The optic according to claim 6 wherein the periodic peripheral edge surface is flat.

8. The optic according to claim 7 wherein the flat periodic peripheral edge surface is parallel to a central optic axis of the optic.

9. The optic according to claim 8 wherein the flat periodic peripheral edge surface includes alternating troughs and crests defined by radii substantially smaller than an optic radius.

10. An eye implantable intraocular lens comprising:
an optic adapted for placement in a capsular bay of the eye and for directing light toward an eye retina, the optic having a central optical axis, an anterior surface, a posterior surface and a substantially flat undulating peripheral edge surface, the undulated peripheral edge surface intersecting at least one of the anterior and posterior surface to form a discontinuous shape corner edge between the undulating surface and at least one of the anterior and posterior surfaces; and
at least one fixture member for securing the optic within the capsular bay.

11. The optic according to claim 10 wherein the undulated peripheral edge surface is flat.

12. The optic according to claim 11 wherein the flat undulated peripheral edge surface is parallel to a central optic axis of the optic.

13. The optic according to claim 12 wherein the flat undulated peripheral edge surface includes alternating troughs and crests defined by radii substantially smaller than an optic radius.

14. An eye implantable intraocular lens comprising:
an optic adapted for placement in a capsular bay of the eye and for directing light toward an eye retina, the optic having a central optical axis, an anterior surface, a posterior surface and a substantially flat periodical peripheral edge surface, the undulated peripheral surface intersecting at least one of the anterior and posterior surface to form a discontinuous shape corner edge between the periodical peripheral edge surface and at least one of the anterior and posterior surfaces; and
at least one fixture member for securing the optic within the capsular bay.

15. The optic according to claim 14 wherein the peripheral edge surface is flat.

16. The optic according to claim 15 wherein the flat peripheral edge surface is parallel to a central optic axis of the optic.

17. The optic according to claim 16 wherein the flat peripheral edge surface includes alternating troughs and crests defined by radii substantially smaller than an optic radius.
